(19) United States
(12) Patent Application Publication
(10) Pub. No.: US 2024/0189128 A1
(43) Pub. Date: Jun. 13, 2024

(54) DEVICES, SYSTEMS, AND METHODS FOR IMPLANTING A MEDICAL DEVICE

(71) Applicant: BOSTON SCIENTIFIC SCIMED, INC., MAPLE GROVE, MN (US)

(72) Inventors: Ryan V. Wales, Northborough, MA (US); Kurt Nicholas Robakiewicz, Upton, MA (US); Jeff Gray, Sudbury, MA (US)

(73) Assignee: BOSTON SCIENTIFIC SCIMED, INC., MAPLE GROVE, MN (US)

(21) Appl. No.: 18/536,363

(22) Filed: Dec. 12, 2023

Related U.S. Application Data
(60) Provisional application No. 63/432,265, filed on Dec. 13, 2022.

(51) Int. Cl. A61F 2/962; A61F 2/844; A61F 2/848; A61F 2/852

(52) U.S. Cl. CPC A61F 2/962; A61F 2/844; A61F 2/848; A61F 2/852; A61F 2002/826

(57) ABSTRACT

Devices, systems, and methods for delivering and deploying an implantable medical device with respect to a target site along a tissue wall by applying suction to draw the tissue wall to a delivery and deployment device, such as to a tissue-penetrating end of the delivery and deployment device. An additional implantable device may be deployed distal to the tissue wall, such as to facilitate manipulation of the distal wall. At least a portion of the first implantable medical device may be deployed with respect to the additional implantable medical device, such as within a lumen of the additional implantable medical device. The implantable medical device may shift from a delivery configuration to an expanded deployed configuration, and at least a portion of the implantable medical device may expand beyond the deployed configuration after deployment device to further resist migration of the implantable medical device from the target site.

### CROSS-REFERENCE TO RELATED APPLICATIONS

This application claims the benefit of priority under 35 U.S.C. § 119 to U.S. Provisional Application No. 63/432,265, filed Dec. 13, 2022, the entire disclosure of which is hereby incorporated by reference herein for all purposes.

### FIELD

The present disclosure relates generally to devices, systems, and methods used in performing procedures within a body. More particularly, the present disclosure relates generally to devices, systems, and methods used to access an anatomical site within a patient's body, such as via another anatomical site, such as endoscopically. The present disclosure also generally relates to deploying a device at an anatomical site, such as across anatomical structures. The present disclosure further relates to improvements to implantable medical device retention with respect to an anatomical deployment site.

### BACKGROUND

Various medical, surgical, laparoscopic, endoscopic, etc., procedures involve grasping and/or manipulating anatomical structures and/or tissues with respect to one another. For instance, various stents may be used to hold tissue in apposition, and/or to effect (e.g., establish or occlude) connections (e.g., fluid communication) between anatomical structures such as organs, cavities, lumens, passages, etc. In some instances, it is desirable to create a semi-permanent or permanent anastomosis with an implantable medical device to allow fluid flow and/or drainage from one anatomical structure to another anatomical structure. However, medical professionals have faced various challenges in accessing and securing engagement of an anatomical structure in which an implantable medical device is to be deployed and/or in accessing and securing a distal anatomical structure to be held in apposition with a proximal anatomical structure such as by an implantable medical device; in deploying devices across apposed anatomical structures; and in maintaining secure retention of the deployed device in position during the term of treatment (which may, in some cases, be indefinite). It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a delivery and deployment system, for creating a bypass across a proximal anatomical structure and a distal anatomical structure, includes a delivery sheath having a wall defining a lumen therethrough; a tissue-penetrating end at a distal end of the delivery sheath; a suction lumen extending along the delivery sheath and the tissue-penetrating end and fluidly couplable to a suction source to apply suction along the tissue-penetrating end; and a bypass stent deliverable within the delivery sheath, and expandable from a delivery configuration, dimensioned to fit within the delivery sheath, to an expanded configuration larger than the delivery configuration, and configured to extend across the proximal anatomical structure and the distal anatomical structure.

In some aspects, the tissue-penetrating end has a distal end configured to penetrate through tissue, the suction lumen extending adjacent the distal end to draw tissue to the tissue-penetrating end to facilitating penetration therethrough by the distal end.

Optionally, the delivery sheath is proximally retractable with respect to the tissue-penetrating end to deploy an implantable medical device delivered therein.

In some aspects, the suction lumen is defined through the wall of the delivery sheath and through the tissue-penetrating end.

Optionally, the delivery and deployment system further includes a target stent implantable within the distal anatomical structure and defining a target lumen therethrough, the target stent having a wall configured to be penetrated by the tissue-penetrating end to deliver an end of the bypass stent into the target lumen. In some aspects, the bypass stent defines a bypass lumen therethrough and is configured to be deployed with respect to the target stent with the bypass lumen extending transverse to the target lumen. In some aspects, the bypass stent has a distal end and a proximal end; the bypass stent distal end is expandable to form a distal retention member when the bypass stent expands to the expanded configuration; the bypass stent proximal end is expandable to form a proximal retention member when the bypass stent expands to the expanded configuration; and the wall of the target stent is configured to retain the bypass stent distal retention member within the target lumen. In some aspects, the target stent as deployed within the distal anatomical structure along a target site facilitates penetration of the tissue-penetrating end through a wall of the distal anatomical structure along the target site.

In some aspects, the bypass stent has a distal end and a proximal end; the bypass stent distal end is expandable to form a distal retention member when the bypass stent expands to the expanded configuration; and the bypass stent proximal end is expandable to form a proximal retention member when the bypass stent expands to the expanded configuration. Optionally, at least one of the distal retention member or the proximal retention member expands to a further expanded configuration after expansion of the bypass stent to the expanded configuration. Optionally, the at least one of the distal retention member or the proximal retention member comprises a material activatable after deployment of the bypass stent to expand further than the expanded configuration of the bypass stent.

Optionally, the delivery and deployment system of claim 1, further includes an endoscope with a working channel configured to deliver the delivery sheath to the proximal anatomical structure.

In accordance with various principles of the present disclosure, a delivery and deployment device, configured to deploy an implantable medical device at a movable target site, includes a delivery sheath having a wall defining a lumen therethrough; a tissue-penetrating end at a distal end of the delivery sheath; and a suction lumen defined along the delivery sheath and the tissue-penetrating end and fluidly couplable to a suction source.

In some aspects, the tissue-penetrating end has a distal end configured to penetrate through tissue, the suction lumen extending adjacent the distal end to draw tissue to the tissue-penetrating end to facilitate penetration through the tissue by the distal end.

Optionally, the delivery sheath is proximally retractable with respect to the tissue-penetrating end to deploy an implantable medical device delivered therein.

In accordance with various principles of the present disclosure, a method of deploying an implantable medical device includes penetrating through a proximal tissue wall with a tissue-penetrating end of a delivery and deployment device; and applying suction via the delivery and deployment device to draw a distal tissue wall towards the delivery and deployment device.

Optionally, the method further includes applying suction via a suction lumen along the tissue-penetrating end of the delivery and deployment device.

Optionally, the method further includes penetrating the tissue-penetrating end of the delivery and deployment device through the distal tissue wall to deploy an implantable medical device delivered by the delivery and deployment device.

Optionally, the method further includes extending the tissue-penetrating end of the delivery and deployment device through a wall of a target stent deployed distal to the distal tissue wall to deliver a distal end of the implantable medical device into a lumen defined by the target stent.

Optionally, the method further includes delivering the implantable medical device within a delivery sheath of the delivery and deployment device; proximally withdrawing the delivery sheath to allow the implantable medical device to shift from a delivery configuration to an expanded deployed configuration; and, after the implantable medical device has been deployed and is in the expanded deployed configuration, allowing at least a portion of the implantable medical device to further expand beyond the expanded deployed configuration.

In accordance with various principles of the present disclosure, a medical bypass system includes a first implantable medical device and a second implantable medical device. The first implantable device is deployable across a proximal anatomical structure and a distal anatomical structure. The second implantable medical device is deployable within the distal anatomical structure. In some aspects, the second implantable medical device modifies one or more properties of the distal anatomical structure to facilitate grasping thereof and/or access thereto/therethrough. The first implantable medical device is deployable through a wall of the proximal anatomical structure and a proximal wall of the distal anatomical structure to be deployed with respect to the proximal and distal anatomical structures. The first implantable medical device is also deployable with respect to the second implantable medical device distal to the proximal wall of the distal anatomical structure. Optionally, the distal retention member and the proximal retention member extend radially outwardly from the saddle region to define shoulders configured to retain the first implantable medical device in place with respect to a deployment site. Optionally, the first implantable medical device defines a lumen therethrough, a distal retention member, a proximal retention member, and a saddle region therebetween, and defines a lumen therethrough. The second implantable medical device optionally has a wall defining a lumen therethrough. Optionally, the first implantable medical device is deployable with respect to the second implantable medical device with the lumen of the first implantable medical device extending transverse to the lumen of the second medical device. Optionally, the distal end of the first implantable medical device is configured to extend through the wall of the second implantable medical device and into the lumen of the second implantable medical device. Optionally, the distal end of the first implantable medical device expands within the lumen defined by the second implantable medical device to form a distal retention member therein to retain the first implantable medical device with respect to the second implantable medical device.

In accordance with various principles of the present disclosure, an implantable medical device is expandable to shift from a delivery configuration to an expanded deployed configuration upon deployment at a target site. Once the implantable medical device has been deployed, at least a portion of the implantable medical device further expands. In some embodiments, the implantable medical device is maintained in the delivery configuration within a delivery sheath, and is configured to expand into an expanded deployed configuration upon withdrawal of the delivery sheath to unsheathe or expose the implantable medical device. In some embodiments, at least a portion of the implantable medical device further expands beyond the expanded deployed configuration of the implantable medical device. In some embodiments, a material is associated with at least a portion of the implantable medical device expands after deployment of the implantable medical device and upon exposure to a catalyst. The further-expandable portion of the implantable medical device may be along retention members extending radially outwardly at either end of an intermediate saddle region of the implantable medical device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing by the addition of an ′, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a human gastrointestinal system as an example environment in which embodiments of the present disclosure may be applied or used or deployed, with an example of an embodiment of a delivery and deployment system positioned therein to deploy an implantable medical device with an example of an embodiment of a delivery and deployment device formed in accordance with various principles of the present disclosure.

FIG. 2 is a perspective view of a distal end of an example of an embodiment of a delivery and deployment device such as illustrated in FIG. 1.

FIG. 3 is a cross-sectional view along line III-III of FIG. 2.

FIG. 4 is a perspective view of a delivery and deployment device such as illustrated in FIG. 2 with a delivery sheath being withdrawn to deploy an implantable medical device delivered therein.

FIGS. 5A-5E illustrate sequential stages of deploying an example of an embodiment of an implantable medical device with an example of an embodiment of a delivery and deployment system and delivery and an example of an embodiment of a deployment device formed in accordance with various principles of the present disclosure.

FIG. 6A and FIG. 6B illustrate deployment of an example of an embodiment of a first implantable medical device with respect to an example of an embodiment of a second implantable medical device in accordance with various principles of the present disclosure.

FIG. 7A illustrates an initial deployed configuration of an example of an embodiment of an implantable medical device formed in accordance with various principles of the present disclosure.

FIG. 7B illustrates a further expanded configuration of the implantable medical device illustrated in FIG. 7A

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

Various procedures involve implanting an implantable medical device within a patient. For instance, various procedures involve implanting an implantable medical device across adjacent, typically apposed, anatomical structures. Endoscopic procedures may be preferred over open surgical (involving cutting open the patient's body to gain access into the patient's body and internal anatomical structures) or laparoscopic (typically involving smaller incisions to gain access into the patient's body and internal anatomical structures) procedures for various reasons. For instance, open surgical or even laparoscopic procedures may involve longer recovery times than endoscopic methods which do not involve incisions, but which, instead, typically access the internal anatomy through a natural orifice and advancing equipment, tools, devices, etc., transluminally to the target area. However, endoscopic procedures may pose further challenges with regard to grasping and accessing a target site into which an implantable medical device is to be deployed, particularly if the target site is movable.

For instance, gastrointestinal (GI) procedures, such as gastric bypass surgery, use a lumen-apposing stent to create a gastrojejunostomy between the stomach and the jejunum to facilitate flow of food particulate, liquid, chyme, etc. (generally, “gastric materials”), from the stomach to the lower GI tract. Gastrojejunostomies may be indicated for a variety of reasons, such as weight loss, treatment of diabetes, diseased sections of the small intestine which may need to be bypassed, gastric outlet obstruction (obstruction of emptying of the stomach into the small intestines via the pylorus, which may be caused by obstruction of the pylorus or the portion of the duodenum in proximity to the pylorus, or other disease states of the stomach, pylorus, duodenum, etc.), etc. When a gastric outlet obstruction is encountered, normally patients will have a medical treatment (without surgery), or, if the medical treatments fail, a surgical treatment. Patients who are elderly or who have comorbidities associated with surgical procedures are generally not considered candidates for surgical procedures. An endoscopic procedure provides an alternative solution to candidates who do not qualify for a surgical procedure. The jejunum may be endoscopically accessed via the patient's stomach (which is accessed via the patient's mouth and esophagus without the need for surgical intervention). The creation of a junction (e.g., anastomosis) between the stomach and small intestine allows the obstruction to be bypassed completely. For instance, an implantable medical device may be deployed across the stomach and a portion of the small intestines to create an anastomosis bypassing the normal path through the pylorus into the small intestines. The implantable medical device may be in the form of a stent, reference being made herein simply to a stent for the sake of convenience and without intent to limit.

A variety of challenges to endoscopic gastric bypass procedures exist. For instance, the small intestines may be difficult to grasp and maneuver endoscopically for a variety of reasons, such as the flexibility and/or slippery nature of the small intestines (which do not hold their shape or position with respect to the stomach), their tendency to move with respect to the stomach, etc. Even if the small intestine is accessed and securely grasped, when the physician attempts to puncture into the small intestine, the small intestine may flex out of the way or even move as a cutting tip is thrust forward to the outside of the wall of the small intestine. Once the portion of the small intestine into which a stent is to be deployed is sufficiently grasped for deployment of the stent, there may be further complications with respect to deployment, such as failed deployment into the desired anatomy (e.g., despite grasping of the anatomical structure, failure of the implantable device from being securely deployed therein); incomplete retention of the deployed device and/or migration or detachment of the deployed device from the deployment site (including partial detachment from one or both of the two apposed structures across which the implantable medical device is deployed); mis-deployment of the device into the wrong distal anatomical structure, etc. The present disclosure provides one or more devices, systems, methods, etc., for improving the existing technology or additional capabilities for deploying an implantable medical device, such as creating an endoscopic ultrasound guided gastroenteric anastomosis (“EUS GGA”), and for improving retention thereof at the deployment site.

In accordance with various principles of the present disclosure, a delivery and deployment system for delivering and deploying an implantable medical device is configured to facilitate access and engagement of the system with a tissue wall with respect to which the implantable medical device is to be deployed. The tissue wall may be of a distal anatomical structure accessed from a proximal anatomical structure. In accordance with various principles of the present disclosure, suction is applied by the delivery and deployment device to draw a tissue wall towards the delivery and deployment device to deploy at least a portion of an implantable medical device with respect to a target site along the tissue wall. Suction may be applied along a delivery sheath within which the implantable medical device is delivered and/or along a tissue-penetrating end which penetrates through the target site to deploy at least a portion of the implantable medical device with respect thereto.

In some instance, suction is applied by the delivery and deployment device to draw a distal anatomical structure towards a proximal anatomical structure to deploy an implantable medical device across such anatomical structures. For instance, to create a gastric bypass, a delivery and deployment device is delivered into the patient's stomach. Ultrasound imaging may be used to locate the targeted portion of the small intestine (e.g., duodenum or jejunum) on the other side of the stomach wall into which the distal end of the implantable medical device is to be deployed (with the proximal end of the implantable medical device deployed with respect to the stomach). The tissue-penetrating end of the delivery and deployment device may be an electrocautery device, a puncture device, cutting device, etc., which is used to extend the distal end of the delivery and deployment device through the stomach wall to access the small intestines outside the stomach. A suction source is fluidly communicated with the distal end of the delivery and deployment device to pull the targeted portion of the small intestine up towards the stomach. In such manner, the medical professional may ensure that movements of the small intestine do not interfere with the medical professional's attempt to gain access to the target site along the small intestines which is being held in place by the suction applied by the delivery and deployment device.

Additionally or alternatively, to facilitate deployment and/or retention of an implantable medical device across a proximal anatomical structure and a distal anatomical structure, an additional device may be deployed in the distal anatomical structure. For instance, in the context of a gastric bypass, because the small intestine is highly mobile within the abdominal cavity, even once the implantable medical device has been deployed across the stomach and the small intestine, retention of the implantable medical device in place with respect to the stomach and the small intestine may present a challenge. Provision of an additional implantable device within the small intestine may facilitate deployment of an implantable medical device across the stomach and the small intestine, as well as retention of the implantable medical device deployed across the stomach and the small intestine.

In accordance with various principles of the present disclosure, an additional implantable medical device is deployed within the small intestine at the target site at which the distal end of the implantable medical device forming the bypass is to be deployed. The additional medical device which is deployed within the small intestines may be a stent. To readily and conveniently differentiate between the implantable medical device which creates the bypass (and which also may be a stent), and the implantable medical device implanted in only the distal anatomical structure (in the case of a gastric bypass, the small intestine), the implantable medical device creating the bypass may be referenced herein as the “bypass stent” for the sake of convenience and without intent to limit, and the implantable medical device in the distal anatomical structure may be referenced herein as the “target stent” for the sake of convenience and without intent to limit.

Provision of a target stent in a distal anatomical structure such as the small intestine provides structural stability to the target site, such as by increasing size, stiffness, rigidity, bulk, heft, and/or other characteristics of the distal anatomical structure enhancing graspability thereof. For instance, a target stent may render the distal anatomical structure more solid and stationary, facilitating deployment of the bypass stent with respect thereto.

Moreover, the increased bulk of the target site caused by the target stent therein increases visibility of the target site at the distal anatomical structure with the imaging systems (fluoroscopy, ultrasound, etc.) used during deployment of the bypass stent. Optionally, the target stent is formed of a material imagable by such imaging systems (e.g., radiopaque markers, bands, and/or radiopaque filler materials) to further facilitate identification and locating of the target site at the distal anatomical structure. Accordingly, the target stent enhances viewability of the distal anatomical structure to identify, locate, etc., the distal anatomical structure and the target site with respect to which the bypass stent is to be deployed.

The increased stiffness imparted by a target stent formed in accordance with various principles of the present disclosure may also provide a more secure deployment site in the distal anatomical structure. For instance, the target stent may facilitate anchoring of a distal end of the bypass stent with respect to the target site. The target stent may have a wall formed from a material which allows the bypass stent to extend therethrough, such as for delivery of the distal end of the bypass into a lumen within the target stent. For instance, the target stent wall may be formed from braided filaments which shift with respect to one another to allow the bypass stent to extend through the wall of the target stent. The target stent wall provides a more rigid structure than tissue wall for anchoring of the distal end of the bypass stent with respect thereto, thereby securing retention of the bypass stent with respect to the target site.

Additionally or alternatively, the implantable medical device may be configured to reconfigure itself once deployed. A bypass stent formed in accordance with various principles of the present disclosure shifts from a compact configuration to an expanded deployed configuration. In the delivery configuration, the bypass stent is sized, shaped, configured, and/or dimensioned to facilitate endoscopic delivery to a target site. For instance, the delivery and deployment device may include a delivery sheath which maintains the bypass stent therein in a compact delivery configuration so that the bypass stent is deliverable within the delivery sheath to a target site. It will be appreciated that terms such as compact, compressed, constrained, restrained, etc., including other grammatical forms thereof, may be used interchangeably herein without intent to limit. In accordance with various principles of the present disclosure, the bypass stent further expands or otherwise increases in size after deployment at the target site. The expansion would occur a sufficient amount of time after deployment to not affect the outer diameter of the implantable medical device during deployment so that no modifications to current deployment devices, such as deployment lumen sizes, would be necessary. The bypass stent would be less likely to migrate because of the increase of its size, increasing the normal force thereof against the deployment site walls and, consequently increasing the frictional force of the bypass stent with respect to the deployment site walls, limiting the ability of the bypass stent to move with respect to the deployment site.

The expansion may be activated in a variety of manners. In some embodiments, a catalyst causes expansion of at least a portion of the bypass stent, such as portion formed of or including an activatable material. For instance, exposure of at least a portion of the bypass stent to water/fluid at the deployment site may cause expansion of at least a portion of the bypass stent. Additionally or alternatively, an additional device may cause expansion of at least a portion of the bypass stent. For instance, an additional device emitting light at wavelengths which induce or activate material growth/expansion of at least a portion of the bypass stent may be advanced to the target site after deployment of the bypass stent. In some embodiments, at least a portion of the target stent is formed of and/or includes a superabsorbent polymer activated by a catalyst (e.g., water or other fluid) to increase in size/volume. The expandable material may be incorporated into the stent (e.g., at least a portion of the stent may be formed of such material), or may be separately formed from and coupled to a portion of the stent (e.g., contained within a pouch, membrane, wall, pocket, etc., permeable to the catalyst which causes expansion of the material).

The expansion may be limited to the retention/anchoring features of the stent without affecting the lumen through the bypass stent so that the desired flow of materials through the bypass stent is not affected by the expansion of a portion thereof. More particularly, the stent may have a distal end and a proximal end with a saddle region therebetween, and a lumen extending through the distal end, the saddle region, and the proximal end. The distal end and the proximal end of the stent extend radially outwardly from the saddle region to define retention features or shoulders which engage the target site to retain/anchor the stent with respect to the target site. The expandable portion of such stent may be limited to one or both of the retention features which already extend radially outwardly from the saddle region, without affecting the inner diameter of the lumen through the bypass stent.

Various embodiments of devices, systems, and methods facilitating access to a deployment site and/or deployment of an implantable medical device and/or retention of such implantable medical device with respect to the deployment site will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers with the addition of a ′, and redundant description is generally omitted for the sake of brevity. Moreover, certain features in one embodiment may be used across different embodiments and are not necessarily individually labeled when appearing in different embodiments.

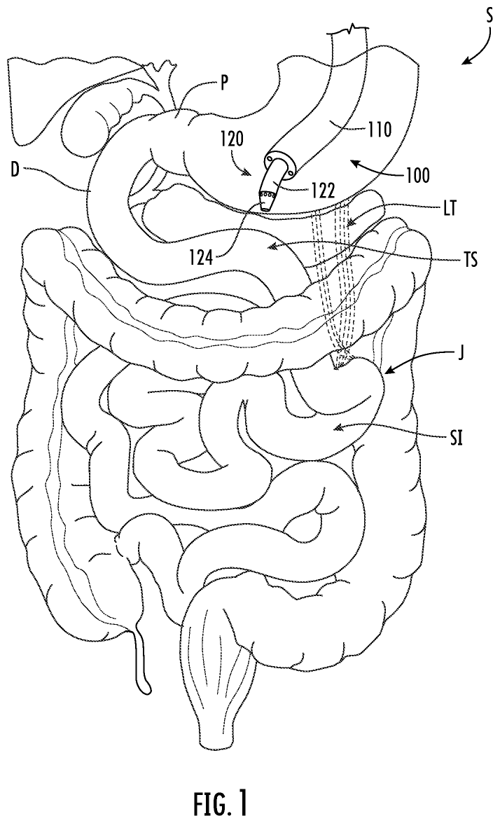

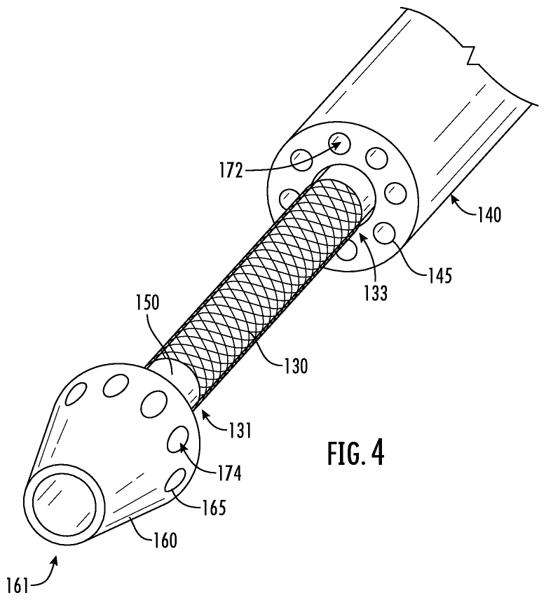

Turning now to the drawings, an example of an embodiment of an environment in which principles of the present disclosure may be applied is a gastrointestinal such as illustrated in FIG. 1. More particularly, a delivery and deployment system 100 formed in accordance with various principles of the present disclosure may be used to deploy an implantable medical device within a gastrointestinal system, such as across a stomach S and a target site TS along the small intestine SI. The target site TS may be along the duodenum D or the jejunum J, depending on the medical needs of the patient. The delivery and deployment system 100 may include an endoscope 110 (or other suitable delivery device, such as a catheter, insertion sheath, etc.) for delivering a delivery and deployment device 120 configured to access the target site TS to deliver and deploy an implantable medical device 130 (illustrated, for example, in FIG. 3 and FIG. 4, and described in further detail below) with respect thereto. The delivery and deployment device 120, or at least a portion thereof, may be delivered within a working channel of the endoscope 100 such as in a manner known to those of ordinary skill in the art.

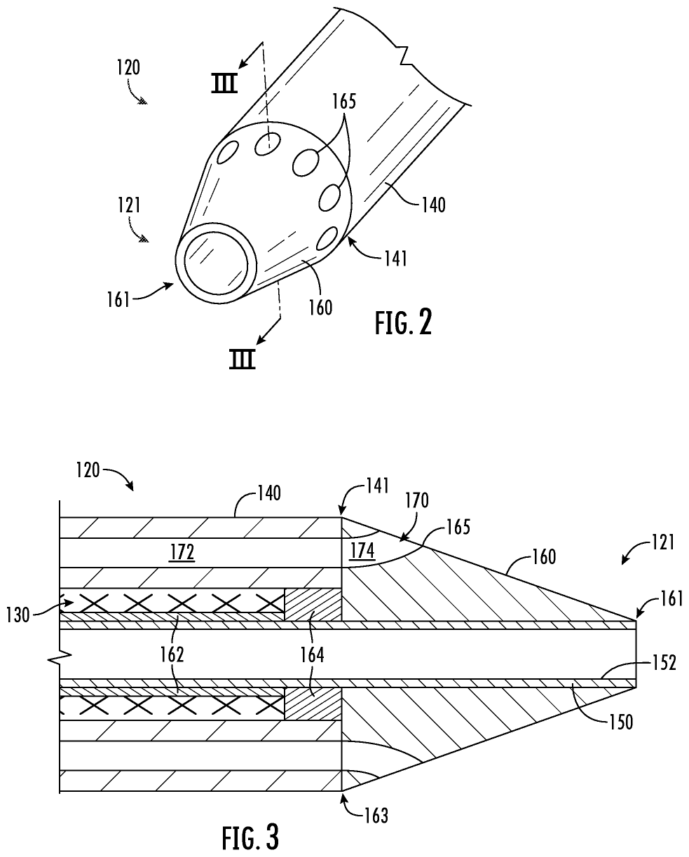

The distal end 121 of the example of an embodiment of a delivery and deployment device 120 illustrated in FIG. 1 is illustrated in further detail in FIG. 2. The illustrated delivery and deployment device 120 includes a flexible tubular element 140 within which an implantable medical device 130 is delivered, as illustrated in the cross-sectional view provided by FIG. 3 along line III-III of FIG. 2. The flexible tubular element 140 is configured to deliver the implantable medical device 130, and may alternatively be referenced herein as a delivery sheath 140. The implantable medical device 130 may be mounted over a delivery shaft 150 within the delivery sheath 140. The implantable medical device 130 may be carried by the delivery shaft 150 and within the delivery sheath 140 for delivery to a deployment site within the patient's body. The delivery shaft 150 may define a guidewire lumen 152 therethrough. A guidewire (such as known to those of ordinary skill in the art, the present disclosure not being limited by the configuration of a guidewire) may be passed through the guidewire lumen 152 so that the delivery and deployment device 120 may be guided over the guidewire into the patient's body and towards the target site TS.

The example of an embodiment of a delivery and deployment device 120 also includes a tissue-penetrating end 160 at the distal end 121 of the delivery and deployment device 120, and, in the example of an embodiment such as illustrated in FIG. 2, along the distal end 141 of the delivery sheath 140. The tissue-penetrating end 160 is configured to penetrate, puncture, cut, or otherwise extend through anatomical tissue, such as the wall of the stomach S and the wall of the small intestine SI. In some embodiments, the tissue-penetrating end 160 has a distal end 161 which is sufficiently sharp to penetrate through anatomical tissue, and may be a knife or other sharp cutting instrument. In other embodiments, the tissue-penetrating end 160 is a cauterization tip (e.g., a monopolar or bi-polar electrocautery tip) with energy supplied thereto by wires 162 coupled to the tissue-penetrating element 160 by wiring connections 164, such as illustrated in FIG. 3.

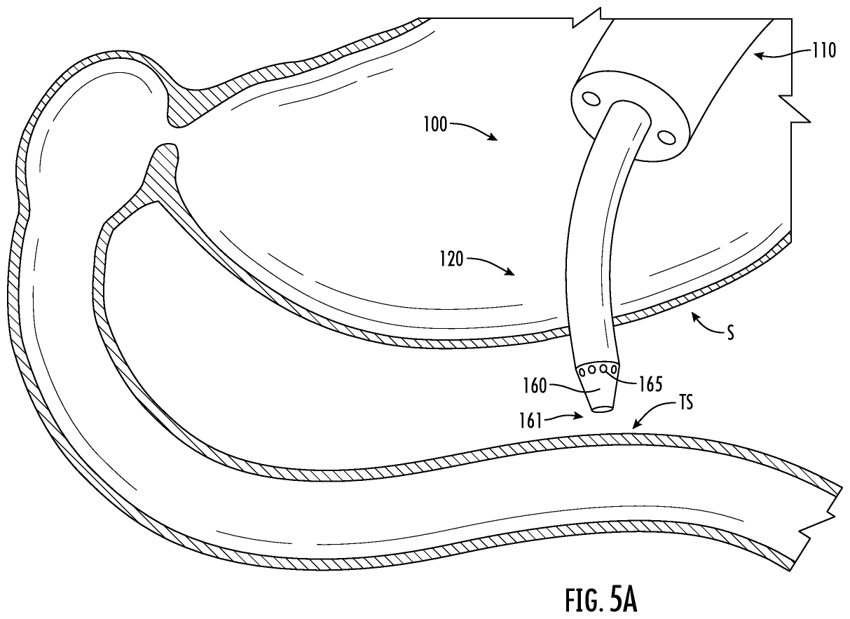

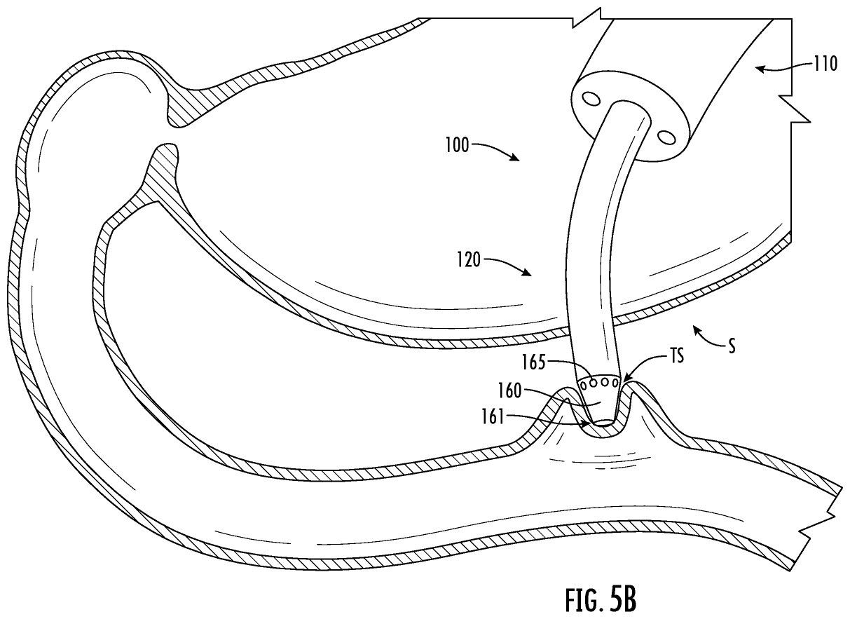

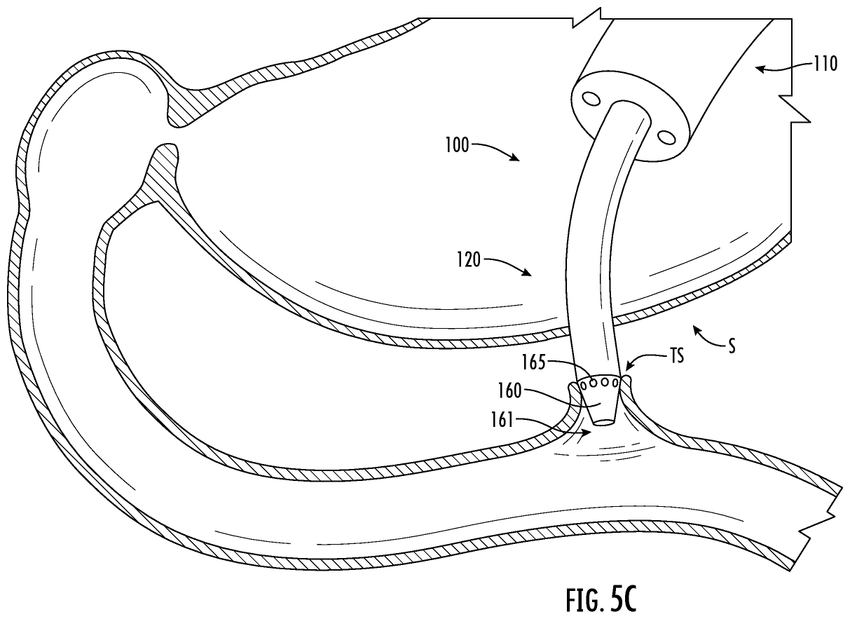

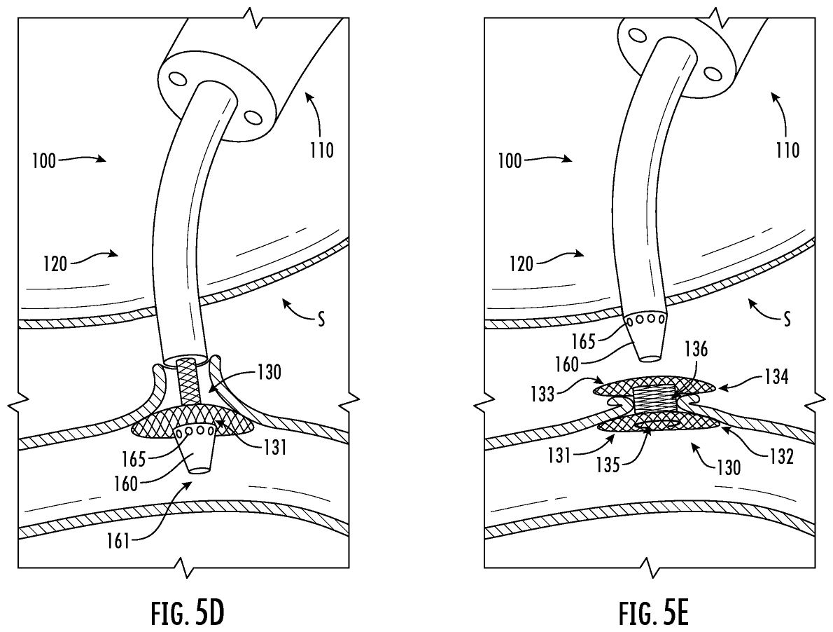

In accordance with various principles of the present disclosure, the delivery and deployment device 120 further includes one or more suction lumens 170 extending along the delivery sheath 140. The one or more suction lumens 170 may be separately formed tubular elements extending along the exterior of the delivery sheath 140 and to the tissue-penetrating end 160. In the example of an embodiment illustrated in FIG. 2, FIG. 3, and FIG. 4, the one or more suction lumens 170 are formed integrally through the delivery sheath 140 and the tissue-penetrating end 160. As may be seen in the cross-sectional view of FIG. 3, and the view of FIG. 4 showing the delivery sheath 140 being withdrawn proximally (prior to expansion of the implantable medical device 130, as described in further detail below), the one or more suction lumens 170 include a first lumen section 172 and a second lumen section 174 in fluid communication with each other. The first lumen section 172 extends through the wall forming the delivery sheath 140 to distal openings 145 in the wall of the delivery sheath 140. The second lumen section 174 extends through the tissue-penetrating end 160 to distal openings 165 through the tissue-penetrating surface of the tissue-penetrating end 160. The one or more suction lumens 170 facilitate positioning of the delivery and deployment device 120 with respect to the target site TS to facilitate deployment of the implantable medical device 130 with respect to the target site TS, as will be appreciated with reference to FIG. 5A, FIG. 5B, FIG. 5C, FIG. 5D, and FIG. 5E, showing an example of an embodiment of a use of a delivery and deployment device 120 formed in accordance with various principles of the present disclosure.

In use, a delivery and deployment system 100, such as formed in accordance with various principles of the present disclosure, is advanced into the patient's body. Advancement of a delivery and deployment system 100 formed in accordance with various principles of the present disclosure is described with respect to a gastrointestinal system, such as illustrated in FIG. 1. However, the present disclosure need not be so limited. If the delivery and deployment system 100 includes an endoscope 110, the endoscope 110 may be positioned in the stomach S to locate the target site TS through the use of an imaging device or system or technique, the present disclosure not being limited in this regard. Once the delivery and deployment system 100 is adjacent the target site TS, the delivery and deployment device 120 may be distally extended out of the endoscope 110 and toward a region of the stomach S adjacent to the target site TS along the small intestine SI. In some embodiments, a smaller cutting device, such as a sharp tissue-penetrating end of a needle, may be advanced into tissue to create an initial entry point for the tissue-penetrating end 160 of the delivery and deployment device 120. The tissue-penetrating end 160 of the delivery and deployment device 120 is then distally extended to penetrate through the stomach S and toward the target site TS, as illustrated in FIG. 5A. A proximal end of the suction lumen 170 is couplable with a suction source (e.g., a vacuum or other suction source such as known to those of ordinary skill in the art). The suction source applies suction through the suction lumen 170 to draw the small intestine SI towards the suction lumen 170 extending from the stomach S, as illustrated in FIG. 5B. The tissue-penetrating end 160 of the delivery and deployment device 120 is further distally advanced to penetrate through the small intestine SI at or adjacent to the target site TS, as illustrated in FIG. 5C. Suction may continue to be applied through the suction lumen 170 to draw the small intestine SI to the tissue-penetrating end 160 as the tissue-penetrating end 160 penetrates therethrough. Once the tissue-penetrating end 160 of the delivery and deployment device 120 is within the small intestine SI, the delivery sheath 140 may be proximally withdrawn to expose the implantable medical device 130 and to commence deployment of the implantable medical device 130, as illustrated in FIG. 5D. The delivery sheath 140 is further proximally withdrawn to deploy the implantable medical device 130, as illustrated in FIG. 5E. The delivery and deployment device 120 may then be further proximally withdrawn from the patient, leaving the implantable medical device 130 deployed across the stomach S and small intestine SI to form a bypass therebetween.

In the illustrated example of an embodiment, the implantable medical device 130 is a stent, and may be alternately referenced herein as a bypass stent 130 for the sake of convenience and without intent to limit. The example of an embodiment of a bypass stent 130 illustrated in FIG. 4, FIG. 5D, and FIG. 5E has a distal end 131 and a proximal end 133, and is generally tubular, defining a bypass lumen 135 therethrough, extending between the distal end 131 and the proximal end 133. In the example of an embodiment of a bypass stent 130 illustrated in FIG. 3, FIG. 4, FIG. 5D, and FIG. 5E, the tubular bypass stent 130 is expandable from a delivery configuration (such as illustrated in FIG. 3 and FIG. 4) to an expanded configuration (such as illustrated in FIG. 5E). The delivery configuration is smaller than the expanded configuration to facilitate endoscopic delivery of the bypass stent 130 to the target site TS. The bypass stent 130 may be self-expanding and optionally formed of shape-memory or heat-formable material (e.g., Nitinol or Elgiloy® or shape memory polymers) so that the bypass stent 130 returns to a pre-shaped expanded configuration from a collapsed configuration upon proximal retraction of the delivery sheath 140 (which maintains/restrains/constrains/compresses the implantable medical device in a delivery configuration therein) to unsheathe (and remove any constraint around) the implantable medical device 130.

As may be appreciated with reference to FIG. 4 and FIG. 5D and FIG. 5E, as the bypass stent 130 shifts from a delivery configuration (dimensioned to fit within the delivery sheath 140) to an expanded deployed configuration, the distal end 131 and proximal end 133 expand radially outwardly to form a distal retention member 132 (depicted in FIG. 5D and FIG. 5E) and a proximal retention member 134 (depicted in FIG. 5E), respectively, defining an intermediate or saddle region 136 therebetween. The retention members 132, 134 are configured to seat against the anatomical walls with respect to which the bypass stent 130 is deployed. and are shaped and/or configured to inhibit movement of the bypass stent 130 with respect to the deployment site to resist migration of the bypass stent 130. The distal retention member 132 may be on or adjacent or along or at the distal end 131 of the bypass stent 130 (in other words, closer to the distal end 131 than to the proximal end 133) of the device, and/or the proximal retention member 134 may be on or adjacent or along or at the proximal end 133 of the bypass stent 130 (in other words, closer to the proximal end 133 than to the distal end 131) of the bypass stent 130. It will be appreciated that terms such as on or adjacent or along or at may be used interchangeably herein without intent to limit unless otherwise stated, and are intended to indicate a general relative spatial relation rather than a precisely limited location. In some embodiments, the retention members 132, 134 are shaped similarly, although such shapes may not have the same dimensions (i.e., the shapes may have different relative dimensions, scales, or proportions). For instance, the retention members 132, 134 may be substantial duplicates of each other, oriented in generally the same direction. Alternatively, in some embodiments, the retention member s 132, 134 may be mirror images (e.g., similar shapes facing in generally opposite directions). In some embodiments, the retention members 132, 134 are not generally the same or symmetrical. For instance, the distal retention member 132 and the proximal retention member 134 may have different shapes, dimensions, relative proportions, etc. In some embodiments, such as illustrated in FIG. 5E, the retention members 132, 134 are double-wall retention members, although a single wall configuration of either of the retention members 132, 134 is within the scope of the present disclosure as well.

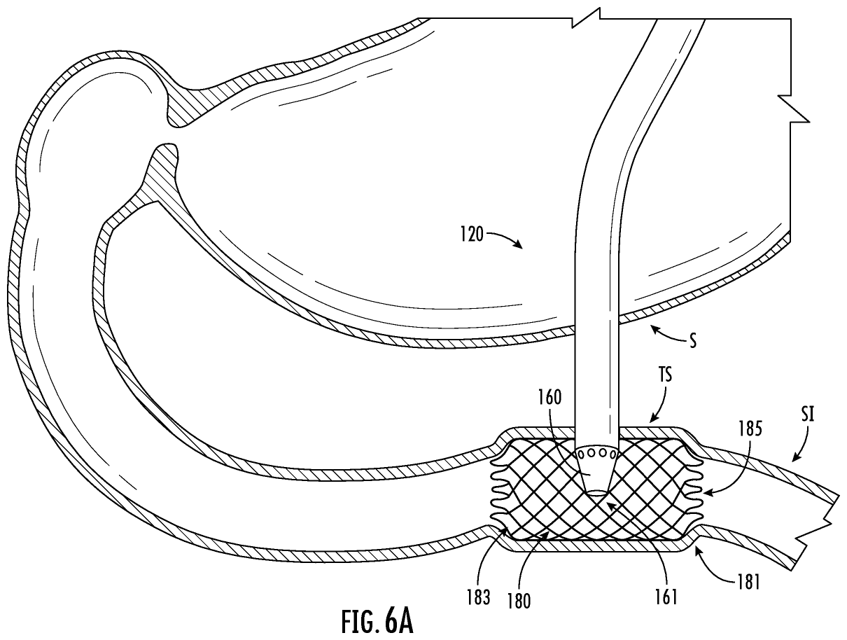

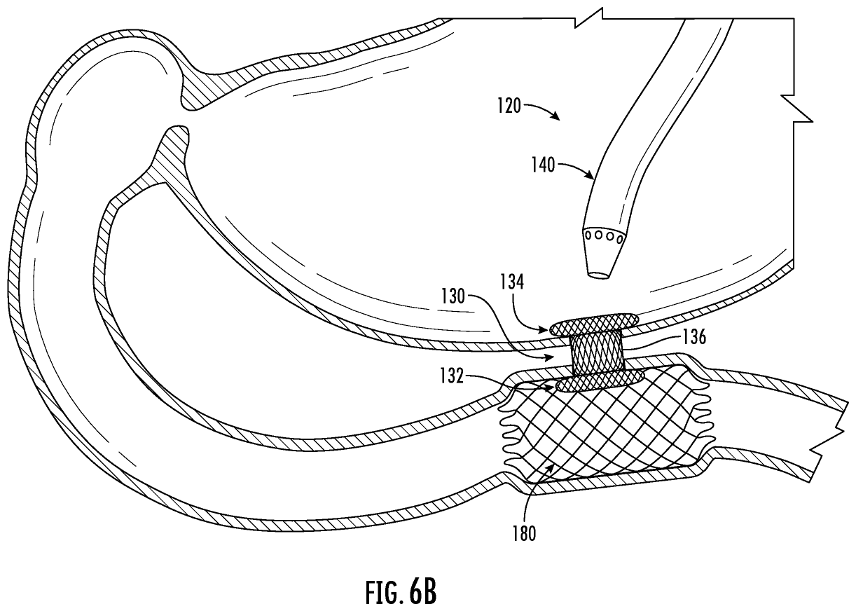

In accordance with various principles of the present disclosure, a delivery and deployment system 100 may include an additional stent 180 (see FIG. 6A) deployed at the deployment site/target site TS. Such additional stent 180 may thus be referenced herein as a target stent 180 for the sake of convenience, and without intent to limit, such as to differentiate from the above-described implantable medical device 130 which may also be a stent and referenced herein as a bypass stent 130. An example of an embodiment of a target stent 130 is illustrated in FIG. 6A and FIG. 6B as having a distal end 181 and a proximal end 183, and having a generally tubular shape defining a target lumen 185 therethrough, extending between the distal end 181 and the proximal end 183. As may be appreciated with reference to FIG. 6A and FIG. 6B, the target stent 180 extends along the interior of the small intestine SI, and is generally coextensive therewith. The target stent 180 is sufficiently rigid to retain its shape within the small intestine SI, yet flexible enough to be grasped or otherwise drawn closer to the stomach S. It will be appreciated, therefore, that a target stent 180 as disclosed herein may be used with a more traditional end effector or grasper such as used to grasp and draw the small intestine SI proximally to the stomach S to create a bypass therebetween. As noted above, provision of such a target stent 180 within the small intestine SI may enhance graspability of the small intestine SI in a variety of manners and/or may enhance visibility of the target site TS, such as with imaging systems.

The target stent 180 is formed to allow the delivery and deployment device 120 to extend therethrough in a direction transverse to the longitudinal extent of the target lumen 185 to extend into the small intestine SI to deploy the bypass stent 130, such as illustrated in FIG. 6A. For instance, the target stent 180 may be formed from one or more members/elements (e.g., wires, strands, filaments, ribbons, etc., all such terms being used interchangeably herein without intent to limit) combined, such as braided, interengaged, intertwined, interwoven, knitted, knotted, looped (e.g., bobbinet-style), weaved, woven, wrapped, or the like to form a rigid and/or semi-rigid tubular scaffold configuration. The filaments are interengaged in a manner which allows the filaments to separate for the tissue-penetrating end 160 to pass therebetween, such as illustrated in FIG. 6A. Furthermore, the filaments preferably are interengaged to return towards one another to retain the deployed distal end 131 of the bypass stent 130 within the target lumen 185 once the delivery and deployment device 120 is proximally withdrawn therefrom (e.g., with the tissue-penetrating end 160 being proximally withdrawn through the expanded bypass lumen 135 of the bypass stent 130), such as illustrated in FIG. 6B. As may further be appreciated with reference to FIG. 6B, the bypass lumen 135 extending through the bypass stent 130 extends generally transverse to the target lumen 185 extending through the target stent 180 to allow materials to be passed from the stomach S into the small intestine SI. Moreover, the distal retention member 132 expands within the target lumen 185, and the filaments of the target stent 180 may add further resistance to the wall of the small intestine SI at the target site TS to enhance retention of the bypass stent 130 with respect to the target site TS.

It will be appreciated that, like the target stent 180, the bypass stent 130 may be formed from one or more members/elements (e.g., struts, wires, strands, filaments, ribbons, etc.) combined (e.g., braided, interengaged, intertwined, interwoven, knitted, knotted, looped (e.g., bobbinet-style), weaved, woven, wrapped, or the like) to form a rigid and/or semi-rigid structure which may be self-expanding from a contracted configuration as described above. Alternatively or additionally, the members forming the bypass stent 130 may be formed by cutting (e.g., by laser-cutting) a tubular structure (e.g., an, optionally monolithic, cylindrical tubular member) into an expandable configuration, the cuts forming members such as strut members. The wall of the bypass stent 130 may have gaps, apertures, openings, interstices, etc., therethrough to facilitate tissue ingrowth with respect to the bypass stent 130. However, the members forming the bypass stent 130 generally are not interengaged to separate from one another as in the target stent 180. A coating material may be applied to, over, on, etc. (such terms being used interchangeably herein without intent to limit) at least a portion of the bypass stent 130 wall to fill in such gaps, apertures, openings, interstices, etc., to inhibit or prevent flow or leakage of materials therethrough and/or to inhibit tissue ingrowth therein. The coating may be any known or heretofore known generally fluid impermeable biocompatible material, including, without limitation, silicone, styrene isoprene butadiene (SIBS), polytetrafluorocthylene (PTFE), expanded polytetrafluorocthylene (cPTFE), ethylene tetrafluoroethylene (ETFE), fluorinated ethylene propylene (FEP), urethane, polyurethane, polyvinylidene chloride (PVC), polyether block amides (PEBA), polyimide, polyethylene, polyethylene terephthalate (PET), polysulfone, nylon, polytrimethylene terephthalate, polyvinylidene difluoride (PVDF), polyester, polyether-ester, polypropylene, polyolefin, polystyrene, polynapthalene, polyethylene napthalate (PEN), polyetherether ketone (PEEK), polyetherimide, polyphenylene sulfide (PPS), polyphenylenc oxide (PPO), perfluoro(propyl vinyl ether) (PFA), polyparaphenylene teraphthalamide, polybutylene terephthalate (PBT), polyoxymethylene (POM), polyether block ester, poly(styrene-butadiene-styrene) (SBS), styrene-cthylene-butylene-styrene (SEBS), poly(styrene-b-isobutylene-b-styrene), ethylene vinyl alcohol, ethylene vinyl acetate copolymers (EVA), polycarbonates, ionomers, thermoplastic elastomers (TPE), epoxy, etc., including copolymers and/or combinations thereof.

The filaments forming either of the stents 130, 180 of the present disclosure may be formed from a variety of non-limiting preferably biocompatible materials, such as, without limitation, a metal, metal alloy, polymer, metal-polymer composite, ceramics, and combinations or subcombinations thereof. For instance, the filaments forming the implantable medical device may be formed from a variety of non-limiting preferably biocompatible polymers, such as, without limitation, polypropylene, polyester, polysulfone, nylon, silicones, polyurethane, polystyrene, polyethylene (PE) (including high-density and low-density PE's), polyethylene terephthalate (PET), polyethylene naphthalate (PEN), polybutylene terephthalate (PBT), polytrimethylene terephthalate, polyether block amides (PEBA), polyetheretherketone (PEEK), polyetherimide (PEI), poly(methyl methacrylate) (PMMA), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, block polyamide/ethers, polyimide (PI), ethylene vinyl alcohol, ethylene vinyl acetate copolymers (EVA), polyphenylene sulfide (PPS), polyphenylene oxide (PPO), poly paraphenylene terephthalamide, perfluoro(propyl vinyl ether) (PFA), polyolefin, epoxy, poly(styrene-b-isobutylene-b-styrene), polycarbonates, ionomers, or the like including mixtures, combinations, subcombinations, and copolymers thereof.

Additionally or alternatively, the members forming the implantable medical device may be formed from a variety of non-limiting preferably biocompatible metals, such as, without limitation, stainless steel, a nickel-titanium alloy such as Nitinol, a nickel-tungsten or tungsten alloy, a cobalt-chromium alloy, a cobalt-chromium-nickel based alloy such as Elgiloy®, a nickel-copper alloy, a nickel-cobalt alloy, a nickel-iron alloy, a nickel-chromium alloy, a nickel-molybdenum alloy, a nickel-chromium-molybdenum alloy, a nickel-cobalt-chromium-molybdenum alloy, a cobalt-chromium-molybdenum alloy, platinum enriched stainless steel, titanium, or the like, including combinations and subcombinations and other alloys thereof. Additionally or alternatively, the members forming an implantable medical device of stent 130, 180 of the present disclosure may be formed from a variety of non-limiting preferably biocompatible natural materials, such as, without limitation, cat or bovine intestine, or the like; a natural fiber, such as silk or cotton, or the like, and combinations and subcombinations thereof. It will be appreciated that the members forming an implantable medical device of stent 130, 180 of the present disclosure may be formed from mixtures, composites, combinations, subcombinations, copolymers, or co-constructions of any of the above.

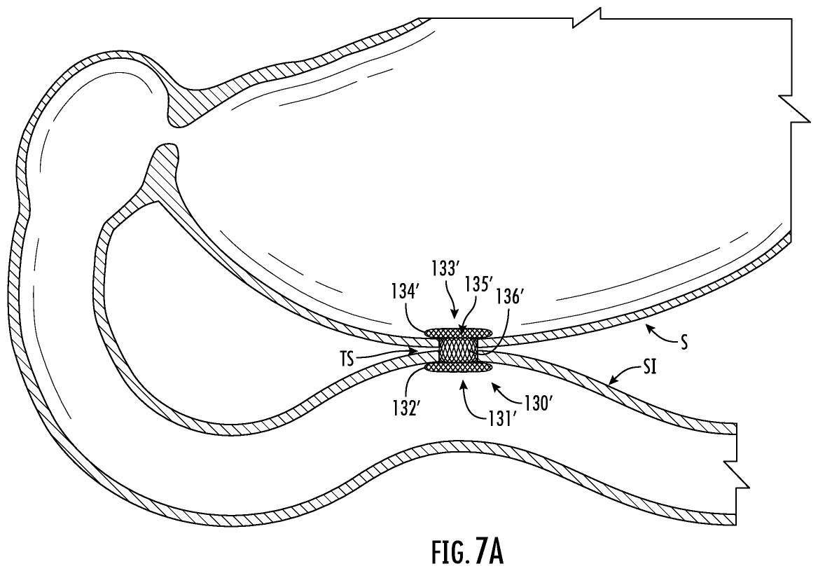

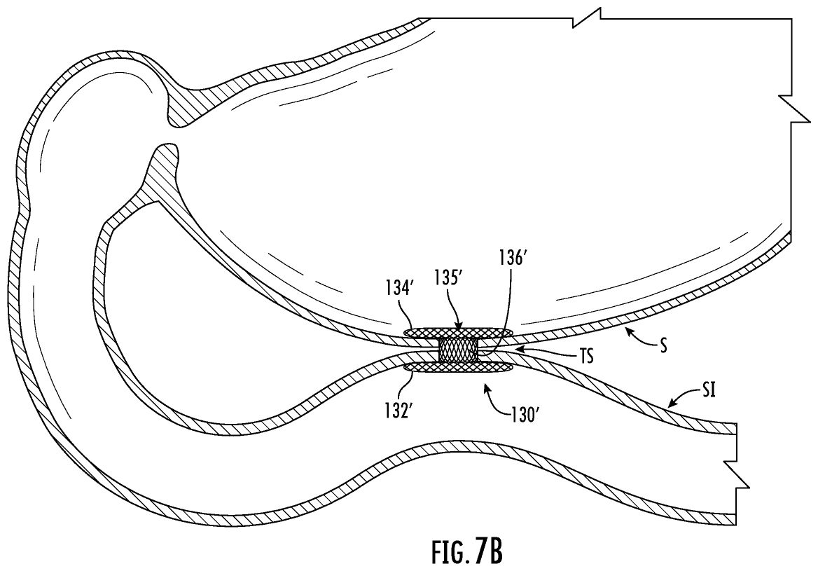

As noted above, the retention members 132, 134 of the bypass stent 130 are configured to expand radially outwardly from the saddle region 136 upon deployment of the bypass stent 130 to hold the bypass stent 130 in place (e.g., anchor) with respect to the target site TS. In accordance with various principles of the present disclosure, the bypass stent 130 may include additional features, associated with at least a portion of the bypass stent 130, to resist migration with respect to the target site TS. Optionally, such features are associated with the retention members 132, 134 in a manner which does not affect the size of the bypass lumen 135. For instance, in the example of an embodiment of a modified bypass stent 130′ illustrated in FIG. 7A and FIG. 7B, the retention members 132′, 134′ are configured to expand further (as illustrated in FIG. 7B) after the initial expansion upon deployment (as illustrated in FIG. 7A). Such further expansion of the retention members 132′, 134′ enhances retention of the distal end 131′ and proximal end 133′ of the bypass stent 130′ with respect to the target site TS, and with respect to the stomach S and small intestine SI in the example of an embodiment illustrated in FIG. 7B. The further expansion may be of a coating applied to at least a portion of the bypass stent 130′, or of a material extruded or overmolded over at least a portion of the bypass stent 130′. For instance, in some embodiments, the expandable material may be coated, extruded, overmolded, etc., over the filaments forming the bypass stent 130′. Additionally or alternatively, in some embodiments, the expandable material may be held within at least a portion of the bypass stent 130′, such as within a permeable container within one or both of the retention members 132′, 134′.

The further expansion of the retention members 132′, 134′ may be activated by a catalyst acting after the bypass stent 130′ has completed shifting from its delivery configuration to its deployed configuration. Activation of the expandable material may be in any of a variety of manners, such as, without limitation, via contact with a material such as fluid (e.g., water, saline, etc.), and/or exposure to energy waves (e.g., light, heat, etc.) and/or another catalyst causing expansion of an expandable material. For example, an additional device may be introduced to emit a certain wavelength of light which induces material growth of at least a portion of the retention members 132′, 134′. Additionally or alternatively, a superabsorbent polymer may be incorporated with respect to the retention members 132′, 134′ to allow the retention members 132′, 134′ to grow beyond their nominal deployment size illustrated in FIG. 7A. Optionally, the saddle region 136′ remains unaffected by the additional expansion of the retention members 132′, 134′ so that the bypass lumen 135 maintains its initial deployed configuration. Optionally, such further expansion of the bypass stent 130′ beyond the expanded deployed configuration may be reversible, such as to facilitate removal of the bypass stent 130′.

It will be appreciated that other than the further expansion of portions of the bypass stent 130′ after deployment, the above-described bypass stents 130, 130′ can be arranged and operate in substantially the same or similar manners. Accordingly, for the sake of brevity and convenience, and without intent to limit, common elements with common functions are indicated with the same reference characters differing by addition of a ‘ to the elements of the bypass stent 130’, reference being made to the above descriptions of similar elements and operations described with respect to the bypass stent 130.

In view of the above, it should be understood that the various embodiments illustrated in the figures have several separate and independent features, which each, at least alone, has unique benefits which are desirable for, yet not critical to, the presently disclosed delivery and deployment devices and systems and/or implantable medical devices. Therefore, the various separate features described herein need not all be present in order to achieve at least some of the desired characteristics and/or benefits described herein. Only one of the various features may be present, or one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

It is to be understood by one of ordinary skill in the art that the present discussion is a description of illustrative examples of embodiments only, and is not intended as limiting the broader aspects of the present disclosure. Various further benefits of the various aspects, features, components, and structures of a devices, systems, and methods such as described above, in addition to those discussed above, may be appreciated by those of ordinary skill in the art.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A delivery and deployment system for creating a bypass across a proximal anatomical structure and a distal anatomical structure, said delivery and deployment system comprising:
a delivery sheath having a wall defining a lumen therethrough;
a tissue-penetrating end at a distal end of said delivery sheath;
a suction lumen extending along said delivery sheath and said tissue-penetrating end and fluidly couplable to a suction source to apply suction along said tissue-penetrating end; and
a bypass stent deliverable within said delivery sheath, and expandable from a delivery configuration, dimensioned to fit within said delivery sheath, to an expanded configuration larger than the delivery configuration, and configured to extend across the proximal anatomical structure and the distal anatomical structure.

2. The delivery and deployment system of claim 1, wherein said tissue-penetrating end has a distal end configured to penetrate through tissue, said suction lumen extending adjacent said distal end to draw tissue to said tissue-penetrating end to facilitating penetration therethrough by said distal end.

3. The delivery and deployment system of claim 2, wherein said delivery sheath is proximally retractable with respect to said tissue-penetrating end to deploy an implantable medical device delivered therein.

4. The delivery and deployment system of claim 1, wherein said suction lumen is defined through the wall of said delivery sheath and through said tissue-penetrating end.

5. The delivery and deployment system of claim 1, further comprising a target stent implantable within the distal anatomical structure and defining a target lumen therethrough, said target stent having a wall configured to be penetrated by said tissue-penetrating end to deliver an end of said bypass stent into the target lumen.

6. The delivery and deployment system of claim 5, wherein said bypass stent defines a bypass lumen therethrough and is configured to be deployed with respect to said target stent with the bypass lumen extending transverse to the target lumen.

7. The delivery and deployment system of claim 6, wherein:
said bypass stent has a distal end and a proximal end;
the bypass stent distal end is expandable to form a distal retention member when said bypass stent expands to the expanded configuration;
the bypass stent proximal end is expandable to form a proximal retention member when said bypass stent expands to the expanded configuration; and
the wall of said target stent is configured to retain said bypass stent distal retention member within the target lumen.

8. The delivery and deployment system of claim 5, wherein said target stent as deployed within the distal anatomical structure along a target site facilitates penetration of said tissue-penetrating end through a wall of the distal anatomical structure along the target site.

9. The delivery and deployment system of claim 1, wherein:
said bypass stent has a distal end and a proximal end;
the bypass stent distal end is expandable to form a distal retention member when said bypass stent expands to the expanded configuration; and
the bypass stent proximal end is expandable to form a proximal retention member when said bypass stent expands to the expanded configuration.

10. The delivery and deployment system of claim 9, wherein at least one of said distal retention member or said proximal retention member expands to a further expanded configuration after expansion of said bypass stent to the expanded configuration.

11. The delivery and deployment system of claim 10, wherein said at least one of said distal retention member or said proximal retention member comprises a material activatable after deployment of said bypass stent to expand further than the expanded configuration of said bypass stent.

12. The delivery and deployment system of claim 1, further comprising an endoscope with a working channel configured to deliver said delivery sheath to the proximal anatomical structure.

13. A delivery and deployment device configured to deploy an implantable medical device at a movable target site, said delivery and deployment device comprising:
a delivery sheath having a wall defining a lumen therethrough;
a tissue-penetrating end at a distal end of said delivery sheath; and
a suction lumen defined along said delivery sheath and said tissue-penetrating end and fluidly couplable to a suction source.

14. The delivery and deployment device of claim 13, wherein said tissue-penetrating end has a distal end configured to penetrate through tissue, said suction lumen extending adjacent said distal end to draw tissue to said tissue-penetrating end to facilitate penetration through the tissue by said distal end.

15. The delivery and deployment device of claim 13, wherein said delivery sheath is proximally retractable with respect to said tissue-penetrating end to deploy an implantable medical device delivered therein.

16. A method of deploying an implantable medical device, said method comprising:
penetrating through a proximal tissue wall with a tissue-penetrating end of a delivery and deployment device; and
applying suction via the delivery and deployment device to draw a distal tissue wall towards the delivery and deployment device.

17. The method of claim 16, further comprising applying suction via a suction lumen along the tissue-penetrating end of the delivery and deployment device.

18. The method of claim 16, further comprising penetrating the tissue-penetrating end of the delivery and deployment device through the distal tissue wall to deploy an implantable medical device delivered by the delivery and deployment device.

19. The method of claim 18, further comprising extending the tissue-penetrating end of the delivery and deployment device through a wall of a target stent deployed distal to the distal tissue wall to deliver a distal end of the implantable medical device into a lumen defined by the target stent.

20. The method of claim 16, further comprising:
delivering the implantable medical device within a delivery sheath of the delivery and deployment device;
proximally withdrawing the delivery sheath to allow the implantable medical device to shift from a delivery configuration to an expanded deployed configuration; and
after the implantable medical device has been deployed and is in the expanded deployed configuration, allowing at least a portion of the implantable medical device to further expand beyond the expanded deployed configuration.
